SEQUENCE LISTING (3 sequences)

SEQ ID NO: 1 - protein, 4 aa, Artificial
  synthetic motif
GDSL

SEQ ID NO: 2 - protein, 4 aa, Artificial
  synthetic motif
GRTT

SEQ ID NO: 3 - protein, 4 aa, Artificial
  synthetic motif
ARTT

(19) United States
(12) Patent Application Publication
(10) Pub. No.: US 2009/0311395 A1
(43) Pub. Date: Dec. 17, 2009

(54) ACYL Transferase Useful for Decontamination

(76) Inventors: Marguerite A. Cervin, Redwood City, CA (US); Gregg Whited, Belmont, CA (US)

Correspondence Address: DANISCO US INC.;ATTENTION: LEGAL DEPARTMENT, 925 PAGE MILL ROAD, PALO ALTO, CA, 94304, US

(21) Appl. No.: 12/085,721

(22) PCT Filed: Dec. 8, 2006
(86) PCT No.: PCT/US2006/047022

§ 371 Date: Mar. 4, 2009

Related U.S. Application Data
(63) Continuation-in-part of application No. 10/581,014, filed on Sep. 11, 2007.
(60) Provisional application No. 60/748,782, filed on Dec. 9, 2005.

(51) Int. Cl. A61L 2/18; C12N 9/10; C12N 9/04; A61L 2/00; A61L 2/23; A23B 4/12; A23B 7/10

(52) U.S. Cl. 426/332; 435/193; 435/190; 422/28; 422/29; 426/335

(57) ABSTRACT

The present invention provides an enzyme system that efficiently generates peracetic acid for use in decontamination applications. In preferred embodiments, the present invention provides a system that comprises an ester substrate, a hydrogen peroxide, and at least one acyl transferase. In some particularly preferred embodiments, the system further comprises at least one surfactant. In alternatively preferred embodiments, the present invention provides at least one wild-type and/or variant acyl transferase. The present invention finds particular use in decontamination involving a wide variety of chemical and biological warfare materials, as well as for general surface cleaning and decontamination.

The present application claims priority to currently pending U.S. Prov. Patent Appln. Ser. No. 60/748,782, filed Dec. 9, 2006. The present application is also a Continuation-in-Part of currently pending U.S. patent application Ser. No. 10/581,014, filed May 30, 2006, which claims priority under 35 U.S.C. §371, to WO 05/056782, filed Dec. 3, 2004, which claims priority under 35 U.S.C. §119, to U.S. Prov. Patent Appln. Ser. No. 60/526,724, filed Dec. 3, 2004, now abandoned.

### FIELD OF THE INVENTION

The present invention provides an enzyme system that efficiently generates peracetic acid for use in decontamination applications. In preferred embodiments, the present invention provides a system that comprises an ester substrate, a hydrogen peroxide, and at least one acyl transferase. In some particularly preferred embodiments, the system further comprises at least one surfactant. In alternatively preferred embodiments, the present invention provides at least one wild-type and/or variant acyl transferase. The present invention finds particular use in decontamination involving a wide variety of chemical and biological warfare materials, as well as for general surface cleaning and decontamination.

### BACKGROUND OF THE INVENTION

Peracetic acid is widely accepted as a decontamination/disinfection agent. However, it is a chemical and carries with it all the problems associated with use of chemical reagents. First, it degrades over time and at high temperatures. In addition, for large surface area cleaning/decontamination, large volumes of liquid chemical are required. Furthermore, it cannot be transported easily due to its corrosive action on tanker trucks. In addition, it has a large chemical footprint. Thus, what is needed is a peracetic acid generation system that resolves these storage and transport issues, is active at a broad range of temperatures, and has a small chemical footprint.

### SUMMARY OF THE INVENTION

The present invention provides an enzyme system that efficiently generates peracetic acid in aqueous solution for use in decontamination applications. In preferred embodiments, the present invention provides a system that comprises an ester substrate, a hydrogen peroxide, and at least one acyl transferase. However, it is not intended that the present invention be limited to peracetic acid, as any peracid (e.g., pernonanoic acid, as well as peracids made from long chain fatty acids C10-C18 or longer chains) find use in the present invention. In addition, peracids made from short-chain fatty acids find use in the present invention. Indeed, a variety of peracids find use in the present invention. In some embodiments, the present invention provides an enzyme system with an additional enzyme that forms hydrogen peroxide. In some additional embodiments, the present invention provides enzyme systems that contain additional compounds that generate hydrogen peroxide, including but not limited to such compounds as sodium percarbonate, glucose oxidase, urea, and various others, including but not limited to those described in U.S. patent application Ser. No. 10/581,014. In some preferred embodiments, the ester substrate is a stable, alcohol ester, although it is not intended that the present invention be limited to any particular ester substrate(s). In some particularly preferred embodiments, the present invention provides a system for enzyme-assisted perhydrolysis in aqueous solutions (e.g., more than about 90% water, although it is not intended that the present invention be limited to any particular percentage of water) comprising at least one ester and at least one peroxide. Indeed, it is contemplated that the present invention will find use in various aqueous systems, including those that have a large percentage of water (e.g., more than about 85%, more than about 95% or more than about 95% water), as well as those with lower percentages of water (e.g., less than about 85%).

In some additional particularly preferred embodiments, the system further comprises at least one surfactant. Thus, in some embodiments, the system comprises at least one enzyme, at least one hydrogen peroxide source, and at least one ester substrate in a buffer. In some further embodiments, the system also comprises at least one detergent, while in still further embodiments, the system also comprises at least one surfactant. Thus, various formulations are contemplated to find use in the present invention. In addition, in some embodiments, the present formulations are neutral in pH, but in some particularly preferred embodiments, the enzyme systems also function in alkaline and slightly acidic environments (e.g., pHs from about 6 to about 10).

It is contemplated that the enzyme system of the present invention will find use in various forms, including liquids, granules, foams, emulsions, etc., designed to fit the need at hand. Indeed, it is not intended that the present invention be limited to any particular format. In yet further embodiments, the acyl transferase system of the present invention is used in conjunction with additional enzymes, including but not limited to proteases, amylases, cellulases, etc.

In alternatively preferred embodiments, the present invention provides at least one wild-type and/or variant acyl transferase. In some preferred embodiments, the enzyme(s) also have lipase activity. The present invention finds particular use in decontamination involving a wide variety of chemical and biological warfare materials, as well as for general surface cleaning and decontamination.

In some embodiments, the present invention finds use in decontamination of materials contaminated by various toxic and/or pathogenic entities, including but not limited to toxic chemicals, mustard, VX, B. anthracis spores, Y. pestis, F. tularensis, fungi, and toxins (e.g., botulinum toxin, ricin, mycotoxins, etc.), as well as cells infected with infective virions (e.g., flaviviruses, orthomyxoviruses, paramyxoviruses, arenaviruses, rhabdoviruses, arboviruses, enteroviruses, bunyaviruses, etc.). In some particularly preferred embodiments, the present invention provides a system that is capable of functioning over a wide temperature range (e.g., from about 16° C. to about 60° C.). In yet additional preferred embodiments, the system provides a small chemical footprint and is stable during short and/or long-term storage. Indeed, it is intended that the system of the present invention will find use in numerous applications.

In still further embodiments, the present invention finds use in decontamination of food and/or feed, including but not limited to vegetables, fruits, and other food and/or feed items. Indeed, it is contemplated that the present invention will find use in the surface cleaning of fruits, vegetables, eggs, meats, etc. Indeed, it is intended that the present invention will find use in the food and/or feed industries to remove contaminants from various food and/or feed items. In some particularly preferred embodiments, methods for food and/or feed decontamination set forth by the Food and Drug Administration and/or other food safety entities, as known to those of skill in the art find use with the present invention.

As indicated herein, the present invention provides enzyme systems for generation of peracid in aqueous solution, suitable for use in decontamination. In some embodiments, the system comprises at least one ester substrate, at least one hydrogen peroxide source, and at least one acyl transferase enzyme. In some preferred embodiments, the peracid is selected from peracetic acid, pernonanoic acid, perproprionic, perbutanoic, perpentanoic, perhexanoic acid, peracids made from long chain fatty acids, and peracids made from short chain fatty acids. In some alternative preferred embodiments, the system further comprises at least one chemical hydrogen peroxide generation system, wherein the chemical hydrogen peroxide generation system comprises at least one chemical selected from sodium percarbonate, perborate, and urea hydrogen peroxide. In some embodiments, the system further comprises at least one enzymatic hydrogen peroxide generation system selected from oxidases and their corresponding substrates. In some additional preferred embodiments, the system further comprises at least one enzymatic hydrogen peroxide generation system, wherein the enzymatic hydrogen peroxide generation system comprises at least one enzyme selected from glucose oxidase, sorbitol oxidase, hexose oxidase, choline oxidase, alcohol oxidase, glycerol oxidase, cholesterol oxidase, pyranose oxidase, carboxyalcohol oxidase, L-amino acid oxidase, glycine oxidase, pyruvate oxidase, glutamate oxidase, sarcosine oxidase, lysine oxidase, lactate oxidase, vanillyl oxidase, glycolate oxidase, galactose oxidase, uricase, oxalate oxidase, xanthine oxidase, and wherein said the enzymatic hydrogen peroxide generating system further comprises at least one suitable substrate for the at least one enzyme. In some still additional embodiments, the system further comprises at least one additional enzyme. In some preferred embodiments, the at least one additional enzyme is selected from proteases, cellulases, amylases, and microbial cell wall degrading enzymes. In some further embodiments, the at least one ester substrate is an alcohol ester. In some yet additional embodiments, the system further comprises at least one surfactant. In some preferred embodiments, the system further comprises at least one detergent. In some additional embodiments, the system is in a form selected from liquids, granules, foams, and emulsions.

The present invention also provides methods for decontamination comprising the steps of: providing an item in need of decontamination, and at least one system for generation of peracid in aqueous solution, suitable for use in decontamination; and exposing the item to the system under conditions such that the item is decontaminated. In some embodiments, the exposing comprises exposing the item to the system under alkaline or acid pH conditions. In some alternative embodiments, the exposing comprises exposing the item to the system under neutral pH conditions. In some still further embodiments, the exposing comprises exposing the item at high temperature. In some preferred embodiments, the high temperature is about 60° C. or higher. However, it is not intended that the present invention be limited to any particular temperature, as various temperatures find use in the methods of the present invention. In some embodiments, the system is in a form selected from liquids, granules, foams, and emulsions. In some yet further preferred embodiments, the system comprises at least one ester substrate, at least one hydrogen peroxide source, and at least one acyl transferase. In some particularly preferred embodiments, the peracid is selected from peracetic acid, pernonanoic acid, perproprionic, perbutanoic, perpentanoic, perhexanoic acid, peracids made from long chain fatty acids, and peracids made from short chain fatty acids. In some alternative preferred embodiments, the method further comprises at least one chemical hydrogen peroxide generation system selected from sodium percarbonate, perborate, and urea hydrogen peroxide. In some additional alternative embodiments, the method further comprises at least one enzymatic hydrogen peroxide generation system selected from oxidases and their corresponding substrates. In some particularly preferred embodiments, the system comprises at least one enzymatic hydrogen peroxide generation system selected from glucose oxidase, sorbitol oxidase, hexose oxidase, choline oxidase, alcohol oxidase, glycerol oxidase, cholesterol oxidase, pyranose oxidase, carboxyalcohol oxidase, L-amino acid oxidase, glycine oxidase, pyruvate oxidase, glutamate oxidase, sarcosine oxidase, lysine oxidase, lactate oxidase, vanillyl, oxidase, glycolate oxidase, galactose oxidase, uricase, oxalate oxidase, xanthine oxidase, and wherein the enzymatic hydrogen peroxide generating system further comprises at least one suitable substrate for the at least one enzyme. In additional embodiments, the method further comprises at least one enzyme or at least one additional enzyme. In some preferred embodiments, the at least one enzyme is selected from proteases, amylases, cellulases, and microbial cell wall degrading enzymes. In some alternative embodiments, the at least one ester substrate is an alcohol ester. In some additional embodiments, the method further comprises at least one surfactant. In some preferred embodiments, decontamination comprises decontaminating items contaminated by at least one toxin and/or at least one pathogen. In some preferred embodiments, the toxin is selected from botulinum toxin, anthracis toxin, ricin, scombroid toxin, ciguatoxin, tetrodotoxin, and mycotoxins. In further preferred embodiments, the pathogen is selected from bacteria, viruses, fingi, parasites, and prions. In some particularly preferred embodiments, the at least one pathogen is selected from Bacillus spp., B. anthracis, Clostridium spp., C. botulinum, C. perfringens, Listeria spp., Staphylococcus spp., Streptococcus spp., Salmonella spp., Shigella ssp., E. coli, Yersinia spp., Y. pestis, Francisella spp., F. tularensis, Camplyobacter ssp., Vibrio spp., Brucella spp., Cryptosporidium spp., Giardia spp., Cyclospora spp., and Trichinella spp. In still further preferred embodiments, the item in need of decontamination is selected from hard surfaces, fabrics, food, feed, apparel, rugs, carpets, textiles, medical instruments, and veterinary instruments. In some particularly preferred embodiments, the food is selected from fruits, vegetables, fish, seafood, and meat. In some still further preferred embodiments, the hard surfaces are selected from household surfaces and industrial surfaces. In some particularly preferred embodiments, the household surfaces are selected from kitchen countertops, sinks, cupboards, cutting boards, tables, shelving, food preparation storage areas, bathroom fixtures, floors, ceilings, walls, and bedroom areas. In some particularly preferred alternative embodiments, the industrial surfaces are selected from food processing areas, feed processing areas, tables, shelving, floors, ceilings, walls, sinks, cutting boards, airplanes, automobiles, trains, and boats.

The present invention also provides methods for decontamination comprising the steps of: providing an item in need of decontamination, and at least one system for generation of peracid in aqueous solution, suitable for use in decontamination; generating the peracid in aqueous solution; and exposing the item to the peracid in aqueous solution under conditions such that the item is decontaminated. In some embodiments, the exposing comprises exposing the item to the system under alkaline or acid pH conditions. In some alternative embodiments, the exposing comprises exposing the item to the system under neutral pH conditions. In some still further embodiments, the exposing comprises exposing the item at high temperature. In some preferred embodiments, the high temperature is about 60° C. or higher. However, it is not intended that the present invention be limited to any particular temperature, as various temperatures find use in the methods of the present invention. In some embodiments, the system is in a form selected from liquids, granules, foams, and emulsions. In some yet further preferred embodiments, the system comprises at least one ester substrate, at least one hydrogen peroxide source, and at least one acyl transferase. In some particularly preferred embodiments, the peracid is selected from peracetic acid, pernonanoic acid, perproprionic, perbutanoic, perpentanoic, perhexanoic acid, peracids made from long chain fatty acids, and peracids made from short chain fatty acids. In some alternative preferred embodiments, the method further comprises at least one chemical hydrogen peroxide generation system selected from sodium percarbonate, perborate, and urea hydrogen peroxide. In some additional alternative embodiments, the method further comprises at least one enzymatic hydrogen peroxide generation system selected from oxidases and their corresponding substrates. In some particularly preferred embodiments, the system comprises at least one enzymatic hydrogen peroxide generation system selected from glucose oxidase, sorbitol oxidase, hexose oxidase, choline oxidase, alcohol oxidase, glycerol oxidase, cholesterol oxidase, pyranose oxidase, carboxyalcohol oxidase, L-amino acid oxidase, glycine oxidase, pyruvate oxidase, glutamate oxidase, sarcosine oxidase, lysine oxidase, lactate oxidase, vanillyl oxidase, glycolate oxidase, galactose oxidase, uricase, oxalate oxidase, xanthine oxidase, and wherein the enzymatic hydrogen peroxide generating system further comprises at least one suitable substrate for the at least one enzyme. In additional embodiments, the method further comprises at least one enzyme or at least one additional enzyme. In some preferred embodiments, the at least one enzyme is selected from proteases, amylases, cellulases, and microbial cell wall degrading enzymes. In some alternative embodiments, the at least one ester substrate is an alcohol ester. In some additional embodiments, the method further comprises at least one surfactant. In some preferred embodiments, decontamination comprises decontaminating items contaminated by at least one toxin and/or at least one pathogen. In some preferred embodiments, the toxin is selected from botulinum toxin, anthracis toxin, ricin, scombroid toxin, ciguatoxin, tetrodotoxin, and mycotoxins. In further preferred embodiments, the pathogen is selected from bacteria, viruses, fungi, parasites, and prions. In some particularly preferred embodiments, the at least one pathogen is selected from Bacillus spp., B. anthracis, Clostridium spp., C. botulinum, C. perfringens, Listeria spp., Staphylococcus spp., Streptococcus spp., Salmonella spp., Shigella ssp., E. coli, Yersinia spp., Y. pestis, Francisella spp., F. tularensis, Camplyobacter ssp., Vibrio spp., Brucella spp., Cryptosporidium spp., Giardia spp., Cyclospora spp., and Trichinella spp. In still further preferred embodiments, the item in need of decontamination is selected from hard surfaces, fabrics, food, feed, apparel, rugs, carpets, textiles, medical instruments, and veterinary instruments. In some particularly preferred embodiments, the food is selected from fruits, vegetables, fish, seafood, and meat. In some still further preferred embodiments, the hard surfaces are selected from household surfaces and industrial surfaces. In some particularly preferred embodiments, the household surfaces are selected from kitchen countertops, sinks, cupboards, cutting boards, tables, shelving, food preparation storage areas, bathroom fixtures, floors, ceilings, walls, and bedroom areas. In some particularly preferred alternative embodiments, the industrial surfaces are selected from food processing areas, feed processing areas, tables, shelving, floors, ceilings, walls, sinks, cutting boards, airplanes, automobiles, trains, and boats.

### DESCRIPTION OF THE FIGURES

FIG. 1 provides a graph showing the enzymatic generation of peracetic acid from hydrogen peroxide or percarbonate.

FIG. 2 provides a graph showing the generation of peracetic acid from glucose and propyleneglycol diacetate.

FIG. 3 provides a graph showing the generation of peracetic acid at three different temperatures (21° C., 40° C., and 60° C.).

FIG. 4 provides a graph showing the ability of the acetyl transferase enzyme to produce concentrated peracetic acid.

### DESCRIPTION OF THE INVENTION

The present invention provides an enzyme system that efficiently generates peracetic acid for use in decontamination applications. In preferred embodiments, the present invention provides a system that comprises an ester substrate, a hydrogen peroxide, and at least one acyl transferase. However, it is not intended that the present invention be limited to peracetic acid, as any peracid (e.g., pernonanoic acid, as well as peracids made from long chain fatty acids C10-C18 or longer chains) find use in the present invention. Indeed, a variety of peracids find use in the present invention. In some particularly preferred embodiments, the system further comprises at least one surfactant. In alternatively preferred embodiments, the present invention provides at least one wild-type and/or variant acyl transferase. The present invention finds particular use in decontamination involving a wide variety of chemical and biological warfare materials, as well as for general surface cleaning and decontamination.

The present invention provides numerous advantages over currently used methods that utilize peracid for cleaning, disinfection and/or decontamination. For example, the present invention facilitates the rapid generation of peracids in situ. In addition, the careful sequential addition of ingredients, peracid extraction, and removal of enzymes by filtration typical of current methods are avoided by the present invention.

While straight (i.e., undiluted) peracetic acid is useful for spore killing, it decays, high levels can be corrosive, it is a biohazard, there are transportation issues with the chemical, and there are limits on storage capabilities. Likewise, hydrogen peroxide is useful, but suffers from similar issues as peracetic acid.

The enzymatic decontamination compositions and methods of the present invention provide numerous significant advantages over currently used solvent-based reactive chemistry decontaminants. Some of these advantages include the non-caustic characteristics of the aqueous-based enzyme system, which allows users to safely use the system without fear of injury to themselves, their equipment, or the environment. In addition, there is a reduced logistical burden, as the enzyme systems are easily transported, easy to use, and require the use of much less water than traditional decontamination methods.

Furthermore, with traditional methods, there is a need to collect post decontaminant by-products. In contrast, the surfactants used in the present invention are biodegradable. The peracid decomposes spontaneously to acetic acid or propionic acid, both of which are also biodegradable. In-situ peracid generation in water, which occurs with the enzymatic system of the present invention, is desirable as it is much safer than reactive chemical generation in solvents and requires much less volume. Moreover, enzymatic activation of hydrogen peroxide has a smaller chemical footprint, and the use of enzyme activation can also control the peracid lifetime. In addition, since hydrogen peroxide has a poor shelf life, the use of percarbonate or an equivalent in the present system circumvents this problem, besides avoiding shipping issues associated with hydrogen peroxide. The use of percarbonate, or other hydrogen peroxide generating compound, instead of hydrogen peroxide also offers flexibility in formulation components. In some preferred embodiments, the formulations comprise ingredients that are inactive until activated by exposure to water. Therefore, these formulations are especially suited for being tailored to the type(s) of materials to be decontaminated, formulation compatibility, and the use of additives (as needed) to provide optimal effectiveness. In addition to finding use as liquids, the enzyme system of the present invention also find use as dry and compact products, as well as gels, emulsions, etc. Thus, the present invention provides the desired flexibility of formulation design, such that the formulation chosen for use is the best for that application.

### DEFINITIONS

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionaries of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a,” “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the term “an item in need of decontamination” refers to any thing that needs to be decontaminated. It is not intended that the item be limited to any particular thing or type of item. For example, in some embodiments, the item is a hard surface, while in other embodiments, the item is an article of clothing. In yet additional embodiments, the item is a textile. In yet further embodiments, the item is used in the medical and/or veterinary fields. In some preferred embodiments, the item is a surgical instrument. In further embodiments, the item is used in transportation (e.g., roads, runways, railways, trains, cars, planes, ships, etc.). In further embodiments, the term is used in reference to food and/or feedstuffs, including but not limited to meat, meat by-products, fish, seafood, vegetables, fruits, dairy products, grains, baking products, silage, hays, forage, etc. Indeed, it is intended that the term encompass any item that is suitable for decontamination using the methods and compositions provided herein.

As used herein, the term “decontamination” refers to the removal of contaminants from an item. In some preferred embodiments, decontamination encompasses disinfection, while in other embodiments, the term encompasses sterilization. However, it is not intended that the term be limited to these embodiments, as the term is intended to encompass the removal of inanimate contaminants, as well as microbial contamination. (e.g., bacterial, fungal, viral, prions, etc.).

As used herein, the term “disinfecting” refers to the removal of contaminants from the surfaces, as well as the inhibition or killing of microbes on the surfaces of items. It is not intended that the present invention be limited to any particular surface, item, or contaminant(s) or microbes to be removed.

As used herein, the term “sterilizing” refers to the killing of all microbial organisms on a surface.

As used herein, the term “sporicidal” refers to the killing of microbial spores, including but not limited to fungal and bacterial spores. The term encompasses compositions that are effective in preventing germination of spores, as well as those compositions that render spores completely non-viable.

As used herein, the terms “bactericidal,” “fungicidal,” and “viricidal” refer to compositions that kill bacteria, fungi, and viruses, respectively. The term “microbicidal” refers to compositions that inhibit the growth and/or replication of any microorganisms, including but not limited to bacteria, fungi, viruses, protozoa, rickettsia, etc.

As used herein, the terms “bacteriostatic,” “fungistatic,” and “virostatic” refer to compositions that inhibit the growth and/or replication of bacteria, fungi, and viruses, respectively. The term “microbiostatic” refers to compositions that inhibit the growth and/or replication of any microorganisms, including but not limited to bacteria, fungi, viruses, protozoa, rickettsia, etc.

As used herein, the term “acyl transferase” refers to an enzyme that is capable of catalyzing a reaction that results in the formation of sufficiently high amounts of peracid suitable for applications such as cleaning, bleaching, and disinfecting. In particularly preferred embodiments, the acyl transferase enzymes of the present invention produce very high perhydrolysis to hydrolysis ratios. The high perhydrolysis to hydrolysis ratios of these distinct enzymes makes these enzymes suitable for use in a very wide variety of applications. In additional preferred embodiments, the acyl transferases of the present invention are characterized by having distinct tertiary structure and primary sequence. In particularly preferred embodiments, the acyl transferases of the present invention comprise distinct primary and tertiary structures. In some particularly preferred embodiments, the acyl transferases of the present invention comprise distinct quaternary structure. In some preferred embodiments, the acyl transferase of the present invention is the M. smegmatis acyl transferase (MsAcT), while in alternative embodiments, the acyl transferase is a variant of this acyl transferase, while in still further embodiments, the acyl transferase is a homolog of this acyl transferase. In further preferred embodiments, a monomeric hydrolase is engineered to produce a monomeric or multimeric enzyme that has better acyl transferase activity than the original monomer. However, it is not intended that the present invention be limited to this specific M. smegmatis acyl transferase, specific variants of this acyl transferase, nor specific homologs of this acyl transferase. In some particularly preferred embodiments, the acyl transferase is the wild-type M. smegmatis acyl transferase disclosed and described in WO 05/056782, incorporated herein by reference in its entirety. In some alternative particularly preferred embodiments, the acyl transferase is one of the variant enzymes or homologs disclosed and described in WO 05/056782. In some more particularly preferred embodiments, the variant comprises the substitution S54V of MsAcT (referred to herein as the “S54V variant” or “variant S54V”).

As used herein, the term “multimer” refers to two or more proteins or peptides that are covalently or non-covalently associated and exist as a complex in solution. A “dimer” is a multimer that contains two proteins or peptides; a “trimer” contains three proteins or peptides, etc. As used herein, “octamer” refers to a multimer of eight proteins or peptides.

As used herein, “cleaning compositions” and “cleaning formulations” refer to compositions that find use in the removal of undesired compounds from items to be cleaned, such as fabric, dishes, contact lenses, other solid substrates, hair (shampoos), skin (soaps and creams), teeth (mouthwashes, toothpastes) etc. The term encompasses any materials/compounds selected for the particular type of cleaning composition desired and the form of the product (e.g., liquid, gel, granule, or spray composition), as long as the composition is compatible with the acyl transferase and other enzyme(s) used in the composition. The specific selection of cleaning composition materials are readily made by considering the surface, item or fabric to be cleaned, and the desired form of the composition for the cleaning conditions during use.

The terms further refer to any composition that is suited for cleaning, bleaching, disinfecting, and/or sterilizing any object and/or surface. It is intended that the terms include, but are not limited to detergent compositions (e.g., liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish detergents).

Indeed, the term “cleaning composition” as used herein, includes unless otherwise indicated, granular or powder-form all-purpose or heavy-duty washing agents, especially cleaning detergents; liquid, gel or paste-form all-purpose washing agents, especially the so-called heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, car or carpet shampoos, bathroom cleaners; hair shampoos and hair-rinses; shower gels and foam baths and metal cleaners; as well as cleaning auxiliaries such as bleach additives and “stain-stick” or pre-treat types.

As used herein, the terms “detergent composition” and “detergent formulation” are used in reference to mixtures which are intended for use in a wash medium for the cleaning of soiled objects. In some preferred embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present invention be limited to any particular detergent formulation or composition. Indeed, it is intended that in addition to acyl transferase, the term encompasses detergents that contain surfactants, transferase(s), hydrolytic enzymes, oxido reductases, builders, bleaching agents, bleach activators, bluing agents and fluorescent dyes, caking inhibitors, masking agents, enzyme activators, antioxidants, and solubilizers.

As used herein the term “hard surface cleaning composition,” refers to detergent compositions for cleaning hard surfaces such as floors, walls, tile, bath and kitchen fixtures, and the like. Such compositions are provided in any form, including but not limited to solids, liquids, emulsions, etc.

As used herein, “dishwashing composition” refers to all forms for compositions for cleaning dishes, including but not limited to granular and liquid forms.

As used herein, “fabric cleaning composition” refers to all forms of detergent compositions for cleaning fabrics, including but not limited to, granular, liquid and bar forms.

As used herein, “textile” refers to woven fabrics, as well as staple fibers and filaments suitable for conversion to or use as yarns, woven, knit, and non-woven fabrics. The term encompasses yarns made from natural, as well as synthetic (e.g., manufactured) fibers.

As used herein, “textile materials” is a general term for fibers, yarn intermediates, yarn, fabrics, and products made from fabrics (e.g., garments and other articles).

As used herein, “fabric” encompasses any textile material. Thus, it is intended that the term encompass garments, as well as fabrics, yarns, fibers, non-woven materials, natural materials, synthetic materials, and any other textile material.

As used herein, the term “compatible,” means that the cleaning composition materials do not reduce the enzymatic activity of the acyl transferase to such an extent that the acyl transferase is not effective as desired during normal use situations. Specific cleaning composition materials are exemplified in detail hereinafter.

As used herein, “effective amount of acyl transferase enzyme” refers to the quantity of acyl transferase enzyme necessary to achieve the enzymatic activity required in the specific application (e.g., decontamination). Such effective amounts are readily ascertained by one of ordinary skill in the art and are based on many factors, such as the particular enzyme variant used, the cleaning application, the specific composition of the cleaning composition, and whether a liquid or dry (e.g., granular, bar) composition is required, and the like.

As used herein, “non-fabric cleaning compositions” encompass hard surface cleaning compositions, dishwashing compositions, personal care cleaning compositions (e.g., oral cleaning compositions, denture cleaning compositions, personal cleansing compositions, etc.), and compositions suitable for use in the pulp and paper industry.

As used herein, “oxidizing chemical” refers to a chemical that has the capability of bleaching. The oxidizing chemical is present at an amount, pH and temperature suitable for bleaching. The term includes, but is not limited to hydrogen peroxide and peracids.

As used herein, “acyl” is the general name for organic acid groups, which are the residues of carboxylic acids after removal of the —OH group (e.g., ethanoyl chloride, CH₃CO—Cl, is the acyl chloride formed from ethanoic acid, CH₃COO—H). The names of the individual acyl groups are formed by replacing the “-ic” of the acid by “-yl.”

As used herein, the term “acylation” refers to the chemical transformation which substitutes the acyl (RCO—) group into a molecule, generally for an active hydrogen of an —OH group.

As used herein, the term “transferase” refers to an enzyme that catalyzes the transfer of functional compounds to a range of substrates.

As used herein, “leaving group” refers to the nucleophile which is cleaved from the acyl donor upon substitution by another nucleophile.

As used herein, the term “enzymatic conversion” refers to the modification of a substrate to an intermediate or the modification of an intermediate to an end-product by contacting the substrate or intermediate with an enzyme. In some embodiments, contact is made by directly exposing the substrate or intermediate to the appropriate enzyme. In other embodiments, contacting comprises exposing the substrate or intermediate to an organism that expresses and/or excretes the enzyme, and/or metabolizes the desired substrate and/or intermediate to the desired intermediate and/or end-product, respectively.

As used herein, the phrase “detergent stability” refers to the stability of a detergent composition. In some embodiments, the stability is assessed during the use of the detergent, while in other embodiments, the term refers to the stability of a detergent composition during storage.

As used herein, the phrase, “stability to proteolysis” refers to the ability of a protein (e.g., an enzyme) to withstand proteolysis. It is not intended that the term be limited to the use of any particular protease to assess the stability of a protein.

As used herein, “oxidative stability” refers to the ability of a protein to function under oxidative conditions. In particular, the term refers to the ability of a protein to function in the presence of various concentrations of H₂O₂ and/or peracid. Stability under various oxidative conditions can be measured either by standard procedures known to those in the art and/or by the methods described herein. A substantial change in oxidative stability is evidenced by at least about a 5% or greater increase or decrease (in most embodiments, it is preferably an increase) in the half-life of the enzymatic activity, as compared to the enzymatic activity present in the absence of oxidative compounds.

As used herein, “pH stability” refers to the ability of a protein to function at a particular pH. In general, most enzymes have a finite pH range at which they will function. In addition to enzymes that function in mid-range pHs (i.e., around pH 7), there are enzymes that are capable of working under conditions with very high or very low pHs. Stability at various pHs can be measured either by standard procedures known to those in the art and/or by the methods described herein. A substantial change in pH stability is evidenced by at least about 5% or greater increase or decrease (in most embodiments, it is preferably an increase) in the half-life of the enzymatic activity, as compared to the enzymatic activity at the enzyme's optimum pH. However, it is not intended that the present invention be limited to any pH stability level nor pH range.

As used herein, “thermal stability” refers to the ability of a protein to function at a particular temperature. In general, most enzymes have a finite range of temperatures at which they will function. In addition to enzymes that work in mid-range temperatures (e.g., room temperature), there are enzymes that are capable of working in very high or very low temperatures. Thermal stability can be measured either by known procedures or by the methods described herein. A substantial change in thermal stability is evidenced by at least about 5% or greater increase or decrease (in most embodiments, it is preferably an increase) in the half-life of the catalytic activity of a mutant when exposed to a different temperature (i.e., higher or lower) than optimum temperature for enzymatic activity. However, it is not intended that the present invention be limited to any temperature stability level nor temperature range.

As used herein, the term “chemical stability” refers to the stability of a protein (e.g., an enzyme) towards chemicals that adversely affect its activity. In some embodiments, such chemicals include, but are not limited to hydrogen peroxide, peracids, anionic detergents, cationic detergents, non-ionic detergents, chelants, etc. However, it is not intended that the present invention be limited to any particular chemical stability level nor range of chemical stability.

As used herein, the phrase “alteration in substrate specificity” refers to changes in the substrate specificity of an enzyme. In some embodiments, a change in substrate specificity is defined as a difference between the K_{cat}/Kₘ ratio observed with an enzyme compared to enzyme variants or other enzyme compositions. Enzyme substrate specificities vary, depending upon the substrate tested. The substrate specificity of an enzyme is determined by comparing the catalytic efficiencies it exhibits with different substrates. These determinations find particular use in assessing the efficiency of mutant enzymes, as it is generally desired to produce variant enzymes that exhibit greater ratios for particular substrates of interest. For example, the acyl transferase enzymes of the present invention are more efficient in producing peracid from an ester substrate than enzymes currently being used in decontamination, cleaning, bleaching and disinfecting applications. Another example of the present invention is an acyl transferase with a lower activity on peracid degradation compared to the wild type. Another example of the present invention is a acyl transferase with higher activity on more hydrophobic acyl groups than acetic acid. However, it is not intended that the present invention be limited to any particular substrate composition nor any specific substrate specificity.

As used herein, “surface property” is used in reference to an electrostatic charge, as well as properties such as the hydrophobicity and/or hydrophilicity exhibited by the surface of a protein.

As used herein, the phrase “is independently selected from the group consisting of . . . ” means that moieties or elements that are selected from the referenced Markush group can be the same, can be different or any mixture of elements as indicated in the following example:

As used herein, the terms “purified” and “isolated” refer to the removal of contaminants from a sample. For example, acyl transferases are purified by removal of contaminating proteins and other compounds within a solution or preparation that are not acyl transferases. In some embodiments, recombinant acyl transferases are expressed in bacterial or fungal host cells and these recombinant acyl transferases are purified by the removal of other host cell constituents; the percent of recombinant acyl transferase polypeptides is thereby increased in the sample.

As used herein, the term “derivative” refers to a protein which is derived from a protein by addition of one or more amino acids to either or both the C- and N-terminal end(s), substitution of one or more amino acids at one or a number of different sites in the amino acid sequence, and/or deletion of one or more amino acids at either or both ends of the protein or at one or more sites in the amino acid sequence, and/or insertion of one or more amino acids at one or more sites in the amino acid sequence. The preparation of a protein derivative is preferably achieved by modifying a DNA sequence which encodes for the native protein, transformation of that DNA sequence into a suitable host, and expression of the modified DNA sequence to form the derivative protein.

Related (and derivative) proteins comprise “variant proteins.” In some preferred embodiments, variant proteins differ from a parent protein and one another by a small number of amino acid residues. The number of differing amino acid residues may be one or more, preferably 1, 2, 3, 4, 5, 10, 15, 20, 30, 40, 50, or more amino acid residues. In some preferred embodiments, the number of different amino acids between variants is between 1 and 10. In some particularly preferred embodiments, related proteins and particularly variant proteins comprise at least 35%, 40%, 45%, 50%, 55%, 60%, 65%, 70%, 75%, 80%, 85%, 90%, 95%, 97%, 98%, or 99% amino acid sequence identity. Additionally, a related protein or a variant protein as used herein, refers to a protein that differs from another related protein or a parent protein in the number of prominent regions. For example, in some embodiments, variant proteins have 1, 2, 3, 4, 5, or 10 corresponding prominent regions that differ from the parent protein.

Several methods are known in the art that are suitable for generating variants of the acyl transferase enzymes of the present invention, including but not limited to site-saturation mutagenesis, scanning mutagenesis, insertional mutagenesis, random mutagenesis, site-directed mutagenesis, and directed-evolution, as well as various other recombinatorial approaches.

In particularly preferred embodiments, homologous proteins are engineered to produce enzymes with the desired activity(ies). In some particularly preferred embodiments, the engineered proteins are included within the SGNH-hydrolase family of proteins. In some most preferred embodiments, the engineered proteins comprise at least one or a combination of the following conserved residues: L6, W14, W34, L38, R56, D62, L74, L78, H81, P83, M90, K97, G110, L114, L135, F180, G205. In alternative embodiments, these engineered proteins comprise the GDSL-GRTT and/or ARTT motifs. In further embodiments, the enzymes are multimers, including but not limited to dimers, octamers, and tetramers. In yet additional preferred embodiments, the engineered proteins exhibit a perhydrolysis to hydrolysis ratio that is greater than 1.

An amino acid residue of a acyl transferase is equivalent to a residue of M. smegmatis acyl transferase if it is either homologous (i.e., having a corresponding position in either the primary and/or tertiary structure) or analogous to a specific residue or portion of that residue in M. smegmatis acyl transferase (i.e., having the same or similar functional capacity to combine, react, and/or chemically interact).

In some embodiments, in order to establish homology to primary structure, the amino acid sequence of an acyl transferase is directly compared to the M. smegmatis acyl transferase primary sequence and particularly to a set of residues known to be invariant in all acyl transferases for which sequence is known. After aligning the conserved residues, allowing for necessary insertions and deletions in order to maintain alignment (i.e., avoiding the elimination of conserved residues through arbitrary deletion and insertion), the residues equivalent to particular amino acids in the primary sequence of M. smegmatis acyl transferase are defined. In preferred embodiments, alignment of conserved residues conserves 100% of such residues. However, alignment of greater than 75% or as little as 50% of conserved residues are also adequate to define equivalent residues. In preferred embodiments, conservation of the catalytic serine and histidine residues are maintained.

Conserved residues are used to define the corresponding equivalent amino acid residues of M. smegmatis acyl transferase in other acyl transferases (e.g., acyl transferases from other Mycobacterium species, as well as any other organisms).

In some embodiments of the present invention, the DNA sequence encoding M. smegmatis acyl transferase is modified. In some embodiments, the following residues are modified: Cys7, Asp10, Ser11, Leu12, Thr13, Trp14, Trp16, Pro24, Thr25, Leu53, Ser54, Ala55, Thr64, Asp65, Arg67, Cys77, Thr91, Asn94, Asp95, Tyr99, Val125, Pro138, Leu140, Pro146, Pro148, Trp149, Phe150, Ile153, Phe154, Thr159, Thr186, Ile192, Ile194, and Phe196. However, it is not intended that the present invention be limited to sequence that are modified at these positions. Indeed, it is intended that the present invention encompass various modifications and combinations of modifications.

In additional embodiments, equivalent residues are defined by determining homology at the level of tertiary structure for an acyl transferase whose tertiary structure has been determined by x-ray crystallography. In this context, “equivalent residues” are defined as those for which the atomic coordinates of two or more of the main chain atoms of a particular amino acid residue of the carbonyl hydrolase and M. smegmatis acyl transferase (N on N, CA on CA, C on C, and O on O) are within 0.13 nm and preferably 0.1 nm after alignment. Alignment is achieved after the best model has been oriented and positioned to give the maximum overlap of atomic coordinates of non-hydrogen protein atoms of the acyl transferase in question to the M. smegmatis acyl transferase. As known in the art, the best model is the crystallographic model giving the lowest R factor for experimental diffraction data at the highest resolution available. Equivalent residues which are functionally and/or structurally analogous to a specific residue of M. smegmatis acyl transferase are defined as those amino acids of the acyl transferases that preferentially adopt a conformation such that they either alter, modify or modulate the protein structure, to effect changes in substrate binding and/or catalysis in a manner defined and attributed to a specific residue of the M. smegmatis acyl transferase. Further, they are those residues of the acyl transferase (in cases where a tertiary structure has been obtained by x-ray crystallography), which occupy an analogous position to the extent that although the main chain atoms of the given residue may not satisfy the criteria of equivalence on the basis of occupying a homologous position, the atomic coordinates of at least two of the side chain atoms of the residue lie with 0.13 nm of the corresponding side chain atoms of M. smegmatis acyl transferase.

In some embodiments, some of the residues identified for substitution, insertion or deletion are conserved residues whereas others are not. The acyl transferase mutants of the present invention include various mutants, including those encoded by nucleic acid that comprises a signal sequence. In some embodiments of acyl transferase mutants that are encoded by such a sequence are secreted by an expression host. In some further embodiments, the nucleic acid sequence comprises a homolog having a secretion signal.

Characterization of wild-type and mutant proteins is accomplished via any means suitable and is preferably based on the assessment of properties of interest. For example, pH and/or temperature, as well as detergent and/or oxidative stability is/are determined in some embodiments of the present invention. Indeed, it is contemplated that enzymes having various degrees of stability in one or more of these characteristics (pH, temperature, proteolytic stability, detergent stability, and/or oxidative stability) will find use. In still other embodiments, acyl transferases with low peracid degradation activity are selected.

As used herein, “corresponding to,” refers to a residue at the enumerated position in a protein or peptide, or a residue that is analogous, homologous, or equivalent to an enumerated residue in a protein or peptide.

As used herein, “corresponding region,” generally refers to an analogous position along related proteins or a parent protein.

The terms “nucleic acid molecule encoding,” “nucleic acid sequence encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “analogous sequence” refers to a sequence within a protein that provides similar function, tertiary structure, and/or conserved residues as the protein of interest (i.e., typically the original protein of interest). For example, in epitope regions that contain an alpha helix or a beta sheet structure, the replacement amino acids in the analogous sequence preferably maintain the same specific structure. The term also refers to nucleotide sequences, as well as amino acid sequences. In some embodiments, analogous sequences are developed such that the replacement amino acids result in a variant enzyme showing a similar or improved function. In some preferred embodiments, the tertiary structure and/or conserved residues of the amino acids in the protein of interest are located at or near the segment or fragment of interest. Thus, where the segment or fragment of interest contains, for example, an alpha-helix or a beta-sheet structure, the replacement amino acids preferably maintain that specific structure.

As used herein, “homologous protein” refers to a protein (e.g., acyl transferase) that has similar action and/or structure, as a protein of interest (e.g., an acyl transferase from another source). It is not intended that homologs be necessarily related evolutionarily. Thus, it is intended that the term encompass the same or similar enzyme(s) (i.e., in terms of structure and function) obtained from different species. In some preferred embodiments, it is desirable to identify a homolog that has a quaternary, tertiary and/or primary structure similar to the protein of interest, as replacement for the segment or fragment in the protein of interest with an analogous segment from the homolog will reduce the disruptiveness of the change. In some embodiments, homologous proteins have induced similar immunological response(s) as a protein of interest.

As used herein, “wild-type” and “native” proteins are those found in nature. The terms “wild-type sequence,” and “wild-type gene” are used interchangeably herein, to refer to a sequence that is native or naturally occurring in a host cell. In some embodiments, the wild-type sequence refers to a sequence of interest that is the starting point of a protein engineering project. The genes encoding the naturally-occurring protein may be obtained in accord with the general methods known to those skilled in the art. The methods generally comprise synthesizing labeled probes having putative sequences encoding regions of the protein of interest, preparing genomic libraries from organisms expressing the protein, and screening the libraries for the gene of interest by hybridization to the probes. Positively hybridizing clones are then mapped and sequenced.

The term “recombinant DNA molecule” as used herein refers to a DNA molecule that is comprised of segments of DNA joined together by means of molecular biological techniques.

The term “recombinant oligonucleotide” refers to an oligonucleotide created using molecular biological manipulations, including but not limited to, the ligation of two or more oligonucleotide sequences generated by restriction enzyme digestion of a polynucleotide sequence, the synthesis of oligonucleotides (e.g., the synthesis of primers or oligonucleotides) and the like.

The degree of homology between sequences may be determined using any suitable method known in the art (See e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]).

The phrases “substantially similar and “substantially identical” in the context of at least two nucleic acids or polypeptides typically means that a polynucleotide or polypeptide comprises a sequence that has at least about 40% identity, more preferable at least about 50% identity, yet more preferably at least about 60% identity, preferably at least about 75% identity, more preferably at least about 80% identity, yet more preferably at least about 90%, still more preferably about 95%, most preferably about 97% identity, sometimes as much as about 98% and about 99% sequence identity, compared to the reference (i.e., wild-type) sequence. Sequence identity may be determined using known programs such as BLAST, ALIGN, and CLUSTAL using standard parameters. (See e.g., Altschul, et al., J. Mol. Biol. 215:403-410 [1990]; Henikoff et al., Proc. Natl. Acad. Sci. USA 89:10915 [1989]; Karin et al., Proc. Natl. Acad. Sci. USA 90:5873 [1993]; and Higgins et al., Gene 73:237-244 [1988]). Software for performing BLAST analyses is publicly available through the National Center for Biotechnology Information. Also, databases may be searched using FASTA (Pearson et al., Proc. Natl. Acad. Sci. USA 85:2444-2448 [1988]). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, “equivalent residues” refers to proteins that share particular amino acid residues. For example, equivalent resides may be identified by determining homology at the level of tertiary structure for a protein (e.g., acyl transferase) whose tertiary structure has been determined by x-ray crystallography. Equivalent residues are defined as those for which the atomic coordinates of two or more of the main chain atoms of a particular amino acid residue of the protein having putative equivalent residues and the protein of interest (N on N, CA on CA, C on C and O on O) are within 0.13 nm and preferably 0.1 nm after alignment. Alignment is achieved after the best model has been oriented and positioned to give the maximum overlap of atomic coordinates of non-hydrogen protein atoms of the proteins analyzed. The preferred model is the crystallographic model giving the lowest R factor for experimental diffraction data at the highest resolution available, determined using methods known to those skilled in the art of crystallography and protein characterization/analysis.

### DETAILED DESCRIPTION OF THE PRESENT INVENTION

The present invention provides an enzyme system that efficiently generates peracetic acid in water for use in decontamination applications. In preferred embodiments, the present invention provides a system that comprises an ester substrate, a hydrogen peroxide, and at least one acyl transferase. However, it is not intended that the present invention be limited to peracetic acid, as any peracid (e.g., pernonanoic acid, as well as peracids made from long chain fatty acids C10-C18 or longer chains) find use in the present invention. Indeed, a variety of peracids find use in the present invention. In some particularly preferred embodiments, the system further comprises at least one surfactant. In alternatively preferred embodiments, the present invention provides at least one wild-type and/or variant acyl transferase. The present invention finds particular use in decontamination involving a wide variety of chemical and biological warfare materials, as well as for general surface cleaning and decontamination.

In some embodiments, the present invention finds use in decontamination of materials contaminated with materials including but not limited to toxic chemicals, mustard, VX, B. anthracis spores, Y. pestis, F. tularensis, fungi, and toxins (e.g., botulinum, ricin, mycotoxins, etc.), as well as cells infected with infective virions (e.g., flaviviruses, orthomyxoviruses, paramyxoviruses, arenaviruses, rhabdoviruses, arboviruses, enteroviruses, bunyaviruses, etc.).

In some particularly preferred embodiments, the present invention provides systems that are capable of functioning over wide temperature ranges (e.g., from about 5° C. to about 90° C.; from about 16° C. to about 60° C.; and from about 25° C. to about 100° C.). In yet additional preferred embodiments, the system provides a small chemical footprint and is stable during short and/or long-term storage. Indeed, it is intended that the system of the present invention will find use in numerous applications. It is contemplated that the enzyme system of the present invention will find use in various forms, including liquids, granules, foams, emulsions, etc., designed to fit the need at hand. Indeed, it is not intended that the present invention be limited to any particular format.

In yet further embodiments, the acyl transferase system of the present invention is used in conjunction with additional enzymes, including but not limited to proteases, amylases, etc. Indeed, it is contemplated that various enzymes will find use in conjunction with the present invention, including but not limited to microbial cell wall-degrading and glycoprotein-degrading enzymes, lysozyme, hemicellulases, peroxidases, proteases, cellulases, xylanases, lipases, phospholipases, esterases, cutinases, pectinases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, endoglucanases, PNGases, amylases, etc., as well as or mixtures thereof. In some embodiments, enzyme stabilizers find use in the present invention. It is contemplated that by using combinations of enzymes, there will be a concurrent reduction in the amount of chemicals needed.

In some particularly preferred embodiments, the present invention finds use in the enzymatic generation of peracids from ester substrates and hydrogen peroxide. It is not intended that the present invention be limited to any specific enzyme for the generation of hydrogen peroxide, as any enzyme that generates H₂O₂ and acid with a suitable substrate finds use in the methods of the present invention. For example, lactate oxidases from Lactobacillus species which are known to create H₂O₂ from lactic acid and oxygen find use with the present invention. Indeed, one advantage of the methods of the present invention is that the generation of acid reduces the pH of a basic solution to the pH range in which the peracid is most effective in bleaching (i.e., at or below the pKa). Other enzymes (e.g., alcohol oxidase, ethylene glycol oxidase, glycerol oxidase, amino acid oxidase, etc.) that can generate hydrogen peroxide also find use with ester substrates in combination with the perhydrolase enzymes of the present invention to generate peracids. Enzymes that generate acid from substrates without the generation of hydrogen peroxide also find use in the present invention. Examples of such enzymes include, but are not limited to proteases. Thus, as described herein, the present invention provides definite advantages over the currently used methods and compositions for decontaminant formulation and use, as well as various other applications.

In some preferred embodiments, the substrates are selected from one or more of the following: formic acid, acetic acid, propionic acid, butyric acid, valeric acid, caproic acid, caprylic acid, nonanoic acid, decanoic acid, dodecanoic acid, myristic acid, palmitic acid, stearic acid, and oleic acid.

In addition to the acyl transferase described herein, various hydrolases find use in the present invention, including but not limited to carboxylate ester hydrolase, thioester hydrolase, phosphate monoester hydrolase, and phosphate diester hydrolase which act on ester bonds; a thioether hydrolase which acts on ether bonds; and α-amino-acyl-peptide hydrolase, peptidyl-amino acid hydrolase, acyl-amino acid hydrolase, dipeptide hydrolase, and peptidyl-peptide hydrolase which act on peptide bonds. Such hydrolase(s) find use alone or in combination with perhydrolase. Preferable among them are carboxylate ester hydrolase, and peptidyl-peptide hydrolase. Suitable hydrolases include: (1) proteases belonging to the peptidyl-peptide hydrolase class (e.g., pepsin, pepsin B, rennin, trypsin, chymotrypsin A, chymotrypsin B, elastase, enterokinase, cathepsin C, papain, chymopapain, ficin, thrombin, fibrinolysin, renin, subtilisin, aspergillopeptidase A, collagenase, clostridiopeptidase B, kallikrein, gastrisin, cathepsin D, bromelin, keratinase, chymotrypsin C, pepsin C, aspergillopeptidase B, urokinase, carboxypeptidase A and B, and aminopeptidase); (2) carboxylate ester hydrolase including carboxyl esterase, lipase, pectin esterase, and chlorophyllase; and (3) enzymes having high perhydrolysis to hydrolysis ratios. Especially effective among them are lipases, as well as esterases that exhibit high perhydrolysis to hydrolysis ratios, as well as protein engineered esterases, cutinases, and lipases, using the primary, secondary, tertiary, and/or quaternary structural features of the perhydrolases of the present invention.

The hydrolase is incorporated into the detergent composition as much as required according to the purpose. It should preferably be incorporated in an amount of 0.00001 to 5 weight percent, and more preferably 0.02 to 3 weight percent. This enzyme should be used in the form of granules made of crude enzyme alone or in combination with other enzymes and/or components in the detergent composition. Granules of crude enzyme are used in such an amount that the purified enzyme is 0.001 to 50 weight percent in the granules. The granules are used in an amount of 0.002 to 20 and preferably 0.1 to 10 weight percent. In some embodiments, the granules are formulated so as to contain an enzyme protecting agent and a dissolution retardant material (i.e., material that regulates the dissolution of granules during use).

In addition, oxidases find use in the present invention, including carbohydrate oxidases selected from the group consisting of aldose oxidase (IUPAC classification EC1.1.3.9), galactose oxidase (IUPAC classification EC1.1.3.9), cellobiose oxidase (IUPAC classification EC1.1.3.25), pyranose oxidase (IUPAC classification EC1.1.3.10), sorbose oxidase (IUPAC classification EC1.1.3.11) and/or hexose oxidase (IUPAC classification EC1.1.3.5), glucose oxidase (IUPAC classification EC1.1.3.4) and mixtures thereof. Indeed, it is contemplated that any suitable oxidase that follows the equation:

Enzyme+reduced substrate→oxidized substrate+H₂O₂

find use in the present invention.

Additional components find use in the formulations of the present invention. Although it is not intended that the formulations of the present invention be so limited, various components are described herein. Indeed, while such components are not essential for the purposes of the present invention, the non-limiting list of adjuncts illustrated hereinafter are suitable for use in the instant compositions and may be desirably incorporated in certain embodiments of the invention, for example to assist or enhance cleaning performance, for treatment of the substrate to be cleaned, or to modify the aesthetics of the cleaning composition as is the case with perfumes, colorants, dyes or the like. It is understood that such adjuncts are in addition to the enzymes of the present invention, hydrogen peroxide and/or hydrogen peroxide source and material comprising an ester moiety. The precise nature of these additional components, and levels of incorporation thereof, will depend on the physical form of the composition and the nature of the cleaning operation for which it is to be used. Suitable adjunct materials include, but are not limited to, surfactants, builders, chelating agents, dye transfer inhibiting agents, deposition aids, dispersants, corrosion inhibitors, additional enzymes, and enzyme stabilizers, catalytic materials, bleach activators, bleach boosters, preformed peracids, polymeric dispersing agents, clay soil removal/anti-redeposition agents, brighteners, suds suppressors, dyes, perfumes, structure elasticizing agents, carriers, hydrotropes, processing aids and/or pigments. In addition to the disclosure below, suitable examples of such other adjuncts and levels of use are found in U.S. Pat. Nos. 5,576,282, 6,306,812, and 6,326,348, herein incorporated by reference. The aforementioned adjunct ingredients may constitute the balance of the cleaning compositions of the present invention.

In some embodiments, the enzyme system of the present invention further comprises enzymes that remove any residual peracid and/or H₂O₂ after decontamination has been achieved. Such enzymes include but are not limited to catalases and/or hydrolytic enzymes.

Importantly, the present invention provides means for effective cleaning, bleaching, and disinfecting over broad pH and temperature ranges. In some embodiments, the pH range utilized in this generation is 4-12. In some alternative embodiments, the temperature range utilized is between about 5° and about 90° C. The present invention provides advantages over the presently used systems (See e.g., EP Appln. 87-304933.9) in that bleaching is possible at the optimum pH of peracid oxidation, as well as providing bleaching at neutral pH, acidic pHs, and at low temperatures.

### EXPERIMENTAL

The following Examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); RT (room temperature); rpm (revolutions per minute); H₂O (water); dH₂O (distilled water); HCl (hydrochloric acid); aa (amino acid); bp (base pair); kb (kilobase pair); kD (kilodaltons); gm (grams); μg and ug (micrograms); mg (milligrams); ng (nanograms); μl and ul (microliters); ml (milliliters); mm (millimeters); nm (nanometers); μm and um (micrometer); M (molar); mM (millimolar); μM and uM (micromolar); U (units); V (volts); MW (molecular weight); sec (seconds); min(s) (minute/minutes); hr(s) (hour/hours); MgCl₂ (magnesium chloride); NaCl (sodium chloride); OD₄₂₀ (optical density at 420 nm); PAGE (polyacrylamide gel electrophoresis); EtOH (ethanol); LB (Luria broth); LA (Luria agar); PBS (phosphate buffered saline [150 mM NaCl, 10 mM sodium phosphate buffer, pH 7.2]); SDS (sodium dodecyl sulfate); Tris (tris(hydroxymethyl)aminomethane); w/v (weight to volume); v/v (volume to volume); wt % (weight percent); PAA (peracetic acid); Per (perhydrolase); per (perhydrolase gene); Ms (M. smegmatis); MsAcT (M. smegmatis acyl transferase); S54V variant (M. smegmatis acyl transferase variant comprising the S54V substitution); MS (mass spectroscopy); Dial (Dial Brands, Inc., Scottsdale, Ariz.); Kemira (Kemira Industrial Chemicals, Helsingborg, Sweden); EM Science (EM Science, Gibbston, N.J.); HP (Hewlett-Packard, Palo Alto, Calif.); ICN (ICN Pharmaceuticals, Inc., Costa Mesa, Calif.); Dial (Dial, Corp., Scottsdale, Ariz.); Pierce (Pierce Biotechnology, Rockford, Ill.); Amicon (Amicon, Inc., Beverly, Mass.); ATCC (American Type Culture Collection, Manassas, Va.); Amersham (Amersham Biosciences, Inc., Piscataway, N.J.); Becton Dickinson (Becton Dickinson Labware, Lincoln Park, N.J.); BioRad (BioRad, Richmond, Calif.); Difco (Difco Laboratories, Detroit, Mich.); GIBCO BRL or Gibco BRL (Life Technologies, Inc., Gaithersburg, Md.); MIDI (MIDI Labs, Newark, Del.); Sigma or Aldrich (Sigma-Aldrich Inc., St. Louis, Mo.); Sorvall (Sorvall Instruments, a subsidiary of DuPont Co., Biotechnology Systems, Wilmington, Del.); Agilent (Agilent Technologies, Palo Alto, Calif.); Minolta (Konica Minolta, Ramsey. NJ); and Zeiss (Carl Zeiss, Inc., Thornwood, N.Y.).

### Example 1

### Killing Curve for B. subtilis Spores by Peracetic Acid (PAA)

In this Example, experiments conducted to determine the killing curve of peracetic acid and peracetic acid in conjunction with detergent (commercially available PUREX® [Dial] was used in this Example) for B. subtilis spores. In these experiments, the B. subtilis spores were prepared as known in the art (See e.g., Siccardi et al., J. Bacteriol., 121:13-19 [1975]). Assays were carried out in duplicate in 96-well, round bottomed microtiter plates (Costar) with peracetic acid (32 wt % in acetic acid; Aldrich). The PAA was serially diluted in either 50 mM KPO₄ buffer, pH 7.1 (“Buffer”), or in a 1:500 dilution of Purex (original formula; Dial) in the same buffer (“Buffer+Det”) in a total volume of 50 μl. The amount of PAA added to the assay was 0, 0.4, 4 or 40 mM. A volume of 5 μl of the spore suspension, containing 10⁹-10¹⁰ spores, was then added to each well and the assay incubated for 15 min at RT. Ice cold LB (150 μl) (See e.g., Sambrook et al., “Molecular Cloning: A Laboratory Manual”, Second Edition (Cold Spring Harbor), [1989]) was then added to each well, mixed, and 100 μl transferred to a fresh 96-well plate. Serial dilutions of each solution were made (in a total volume of 100 μl/well). A volume of 5 μl of each dilution was spotted onto LA plates (Sambrook et al., supra), and incubated at 37° C. for 17-24 h. Colonies were counted and the % spore killing was determined relative to the respective controls (buffer alone or buffer+detergent, without peracid). The results are presented in Table 1, as an average of duplicates from one experiment. However, the experiment was done twice in duplicate with similar results. Based on these results, PAA in the range of about 4 to about 40 mM was determined to be sufficient to kill B. subtilis spores in 15 min.

TABLE 1
Killing of B. subtilis Spores by PAA

| [PAA] | Buffer | | Buffer + Detergent | |
|---|---|---|---|---|
| (mM) | Spores/ml | % Spore Killing | Spores/ml | % Spore Killing |
| 0 | 1.8 × 10⁹ | 0 | 1.6 × 10⁹ | 0 |
| 0.4 | 2.4 × 10⁸ | 86.4 | 1.7 × 10⁹ | 0.05 |
| 4 | 1.6 × 10⁸ | 90 | 2.4 × 10⁸ | 86.4 |
| 40 | 0 | 100 | 0 | 100 |

### Example 2

### Enzymatic Generation of PAA

In this Example, three methods for generation of PAA by acyl transferase are described. In one method, at least one acyl transferase (wild-type or variant) is combined with at least one ester substrate, and hydrogen peroxide in a buffer or detergent, with or without one or more surfactants. In an alternative method, at least one acyl transferase (wild-type or variant), at least one ester substrate, and sodium percarbonate (or other source of H₂O₂) are combined in a buffer or detergent, with or without one or more other surfactants. In yet a further method, at least one acyl transferase (wild-type or variant) is combined with glucose oxidase and glucose, in a concentration sufficient to generate an amount of PAA with which to kill spores in buffer or detergent. In some formulations, one or more other surfactants are also included. Other enzymes that generate H₂O₂ also find use in this system, including oxidases, oxidoreductases (e.g., glycerol oxidase or hexose oxidase). In some preferred embodiments, a co-factor independent alcohol oxidase is used.

### Determination of PAA Concentration

In these experiments, methods known in the art were used to determine the concentration of PAA (See e.g., Pinkernell et al., Analyst, 122:567-571 [1997]). In this ABTS assay, 100 μl of the solution to be analyzed was added to 1 ml of 125 mM potassium citrate buffer, pH 5.0, containing 1.0 mM 3-ethylbenzothiazoline-6-sulfonic acid (ABTS) and 50 uM KI, and allowed to incubate at RT for 3 minutes. The absorbance was measured at 420 nm in a HP 8452A Diode Array Spectrophotomer and compared to a standard curve prepared using authentic standard. The enzymatic reactions to form PAA were initiated with addition of enzyme and conducted at RT. Aliquots were withdrawn from the reactions at the indicated times and analyzed for PAA concentration. The sodium percarbonate used in these experiments was obtained from Kemira and the hydrogen peroxide was obtained from EM Science.

Comparison of PAA Enzymatically Generated from H₂O₂ or Sodium Percarbonate

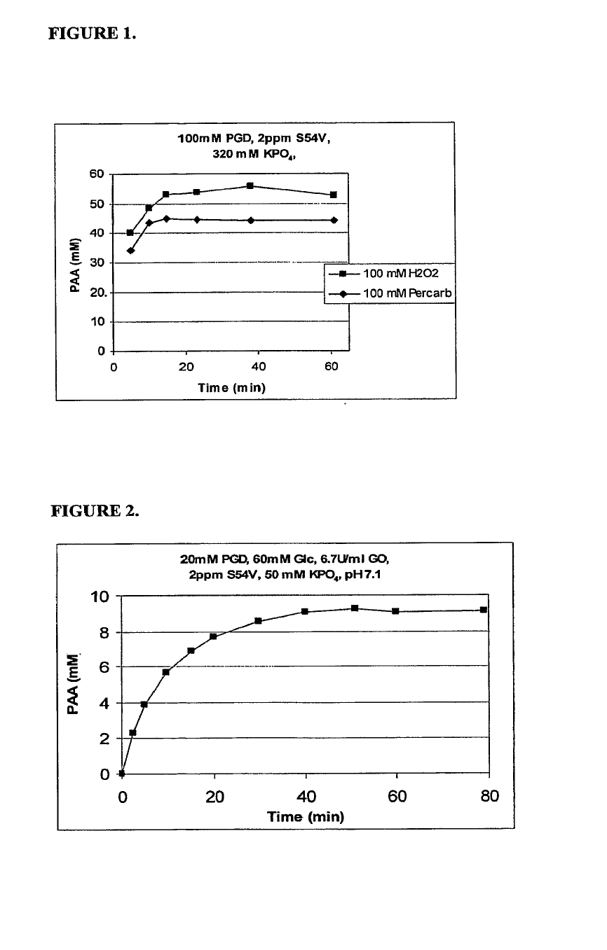

A solution of 39 mM sodium percarbonate (sodium carbonate peroxyhydrate, Technical grade 85%, yielding 100 mM effective H₂O₂; Kemira) was prepared in 320 mM KPO₄ pH 7.1. After dissolution of the solid percarbonate, the resulting solution had a pH of 7.6. To compare the enzymatic production of PAA from prepared H₂O₂ (32 wt %, Aldrich) or H₂O₂ formed from percarbonate under identical pH conditions, two reactions were prepared. One reaction contained 100 mM H₂O₂ in 320 mM KPO₄, pH 7.6, 100 mM 1,2-propylene glycol diacetate (Aldrich), and 2 ppm variant S54V. The absolute concentration of H₂O₂ was assumed from the value stated on the label and not confirmed by analysis. The second reaction contained 39 mM Sodium percarbonate in 320 mM KPO₄, pH 7.1, 100 mM 1,2-propylene glycol diacetate, and 2 ppm S54V. The reactions were initiated by the addition of the enzyme. Samples were withdrawn at the times indicated and the concentration of PAA determined as described in Example 1. The results are shown in FIG. 1. As indicated in this Figure, the progress of the reaction and final concentration of PAA is similar in both cases.

### Example 3

### Enzymatic Generation of PAA Kills B. subtilis Spores

In this Example, experiments conducted to assess the killing ability of enzymatically generated PAA tested with B. subtilis spores are described. Based on the results obtained in the experiments described in Examples 1 and 2, a range of 4 to 40 mM PAA was determined to be sufficient to demonstrate killing of spores of B. subtilis I-168. In these experiments, spore killing was assessed in buffer, as well as in detergent.

### Spore Killing in Buffer

In this experiment, sodium percarbonate was used as the source of H₂O₂. The final solution contained: 100 mM 1,2-propylene glycol diacetate, 2 ppm S54V variant, 39 mM sodium percarbonate (Technical grade 85%; yielding 100 mM effective H₂O₂) in 320 mM KPO₄ pH 7.1 in a total volume of 800 μl. This mix (yield 40 mM PAA) was serially diluted to give additional mixes that yielded 4.9, 9.9 and 20.5 mM PAA. A mix with only 400 mM KPO₄ pH 7.1 was used to determine total spore counts in the absence of PAA. The mixes were allowed to incubate at room temperature for 3 min. A volume of 180 μl of each of the mixes was then dispensed into duplicate wells of a round-bottomed 96-well plate (Costar) that contained 20 μl of the spore suspension used in Example 1, to yield a total volume of 200 μl in each well. The liquid was gently pipetted 4-5 times to ensure mixing of the components. The mixes were incubated with the spores for a further 15 or 30 minutes at room temperature. At the 15 and 30 minute time points, 20 μl were removed from each of the wells, added to wells in a fresh 96-well plate and serially diluted in LB to 10⁻⁷ in a total volume of 100 μl. A volume of 5 μl from each dilution of each spore mixture was spotted onto an LA plate, allowed to dry and then incubated overnight at 37° C. Also at the 15 and 30 min time points, an appropriate volume was removed from each well and diluted sufficiently in dH₂O to yield a measurable amount of PAA using the ABTS assay on scale with a standard, as described in Example 1. Results of these assays are shown in Table 2. The results of the spore killing are presented as an average of the duplicates.

TABLE 2
Use of AcT System to Generate PAA to Kill B. subtilis Spores in Buffer

| | [PAA] | | | [PAA] | | |
|---|---|---|---|---|---|---|
| | Generated | | % Spore | Generated | | % Spore |
| [PAA] (mM) | (mM) | Spores/ml | Killing | (mM) | Spores/ml | Killing |
| (Theoretical) | (15 min) | 15 min | 15 min | (30 min) | 30 min | 30 min |
| 0 | 0 | 1.5 × 10⁹ | 0 | 0 | 1.5 × 10⁹ | 0 |
| 4.9 | 2.2 | 9 × 10⁸ | 40 | 2.4 | 9.4 × 10⁸ | 37 |
| 9.9 | 6.1 | 1 × 10⁸ | 93.3 | 7.1 | 5 × 10⁷ | 96.7 |
| 20.5 | 15 | 4.4 × 10⁴ | 99.997 | 18 | 0 | 100 |
| 39.5 | 35 | 0 | 100 | 41 | 0 | 100 |

### Spore Killing in Detergent.

The experiment was repeated exactly as described except that a 1:500 dilution of Purex in 320 mM KPO₄ pH 7.1, was used in place of the buffer. The results are presented in Table 3 (average of duplicates). Controls included various reaction components in 400 mM KPO₄ pH 7.1 buffer: 2 ppm S54V variant, 2 ppm S54V variant with 39 mM percarbonate, 100 mM 1,2-propylene glycol diacetate, 100 mM 1,2-propylene glycol diacetate with 39 mM sodium percarbonate, 39 mM sodium percarbonate. All these treatments gave equivalent levels of spores/ml after a 30 min incubation, except sodium percarbonate alone (1×10⁹ spores/ml vs 5×10⁹ spores/ml for other controls). This decrease was not seen with sodium percarbonate in combination with other components and was certainly not as dramatic as the killing seen by the mixture of all 3 components at comparable levels (100% killing).

TABLE 3
Use of AcT System to Generate PAA to Kill B. subtilis Spores in Detergent

| | [PAA] | | | [PAA] | | |
|---|---|---|---|---|---|---|
| | Generated | | | Generated | | % Spore |
| [PAA] (mM) | (mM) | Spores/ml | % Spore Killing | (mM) | Spores/ml | Killing |
| (Theoretical) | (15 min) | 15 min | 15 min | (30 min) | 30 min | 30 min |
| 0 | 0 | 2 × 10⁹ | 0 | 0 | 2 × 10⁹ | 0 |
| 4.9 | 3.7 | 2 × 10⁹ | 0 | 3.3 | 5 × 10⁸ | 75 |
| 9.9 | 8.9 | 1.7 × 10⁹ | 91.5 | 8.1 | 4 × 10⁷ | 98 |
| 20.5 | 19.6 | 3 × 10⁵ | 99.99 | 18.9 | 0 | 100 |
| 39.5 | 44.5 | 0 | 100 | 40.4 | 0 | 100 |

### Example 4

### Enzymatic Generation of PAA to Kill Trichoderma reesei Spores

In this Example, experiments conducted to assess the killing ability of PAA on T. reesei spores are described. T. reesei spores were prepared by growing the strain for approximately 4 days on Potato Dextrose (PDA) media at 30° C. When the plate was approximately 75% covered by fungal growth, it was incubated at room temperature for several days until there was confluent growth. The spores were scraped off the plate using a cotton-tipped swab, resuspended in 1 ml of 10% glycerol and frozen at −80° C. until used. Prior to use in the spore killing assay, the spore suspension was thawed, the spores pelleted by centrifugation, washed twice with 1 ml dH₂O, and resuspended in 1 ml of dH₂O. The spore killing experiments were carried out as described in Example 3, except that 20 μl of the fungal spore preparation were added to the wells of the 96-well plate instead of the Bacillus spores. Also, the mixes were made up such that the amount of peracid generated was 40, 13.3, 4.4 and 1.5 mM. The dilutions of the 15 and 30 minute incubations were plated on PDA media. The actual amount of PAA generated was determined as described in Example 1, at the 15 and 30 minute time points. The results are presented in Table 4. These results indicate that fungal spores were killed by PAA generated by the AcT system and at a lower level of PAA than the B. subtilis spores.

TABLE 4
Use of AcT System to Generate PAA to Kill Trichoderma reesei Spores

| [PAA] | [PAA] | | | [PAA] | | |
|---|---|---|---|---|---|---|
| Gen- | Gen- | | % | Gen- | | % |
| erated | erated | Spores/ | Spore | erated | Spores/ | Spore |
| (mM) | (mM) | ml | Killing | (mM) | ml | Killing |
| (Theoretical) | 15 min | 15 min | 15 min | 30 min | 30 min | 30 min |
| 0 | 0 | 2 × 10¹⁰ | 0 | 0 | 2 × 10⁷ | 0 |
| 1.5 | 0.41 | 2 × 10³ | 99.99 | 0.3 | 2 × 10³ | 99.99 |
| 4.4 | 2.1 | 0 | 100 | 2.2 | 0 | 100 |
| 13.3 | 10 | 0 | 100 | 8.5 | 0 | 100 |
| 40 | 42 | 0 | 100 | 38 | 0 | 100 |

### Example 5

### Peracetic Acid Production from Glucose and Propyleneglycol Diacetate

In this Example, experiments to assess the amount of peracetic acid produced from glucose and propyleneglycol diacetate are described. A 15 ml solution was prepared containing 50 mM KPO₄ pH 7.1 with 60 mM glucose, and 20 mM 1,2-propylene glycol diacetate (Aldrich). The solution was continuously sparged with air and stirred at room temperature. The reaction to generate H₂O₂ was initiated by the addition of 100 Units of glucose oxidase (Oxygen HP, Genencor International) and allowed to proceed for 1 hr. A sample was withdrawn and tested for PAA before the addition of 2 ppm S54V variant, to initiate the production of PAA from the formed H₂O₂. The results are presented in FIG. 2. Further samples were withdrawn at the times indicated in FIG. 2, and the concentration of PAA determined as described above. No PAA was detected before the addition of enzyme and approximately 9.5 mM PAA was produced from the 20 mM 1,2-propylene glycol diacetate and the H₂O₂ produced from the glucose/glucose oxidase reaction.

### Example 6

### Generation of Peracetic Acid by AcT at Different Temperatures

In this Example, experiments conducted to assess the generation of peracetic acid by AcT at different temperatures are described. Such generation provides means to resolve problems associated with storage instability of PAA at various temperatures. In these experiments, AcT is used to generate PAA over a range of temperatures from about 20° C. to about 60° C. In some experiments, temperatures such as 21° C., 40° C., and 60° C. are used.

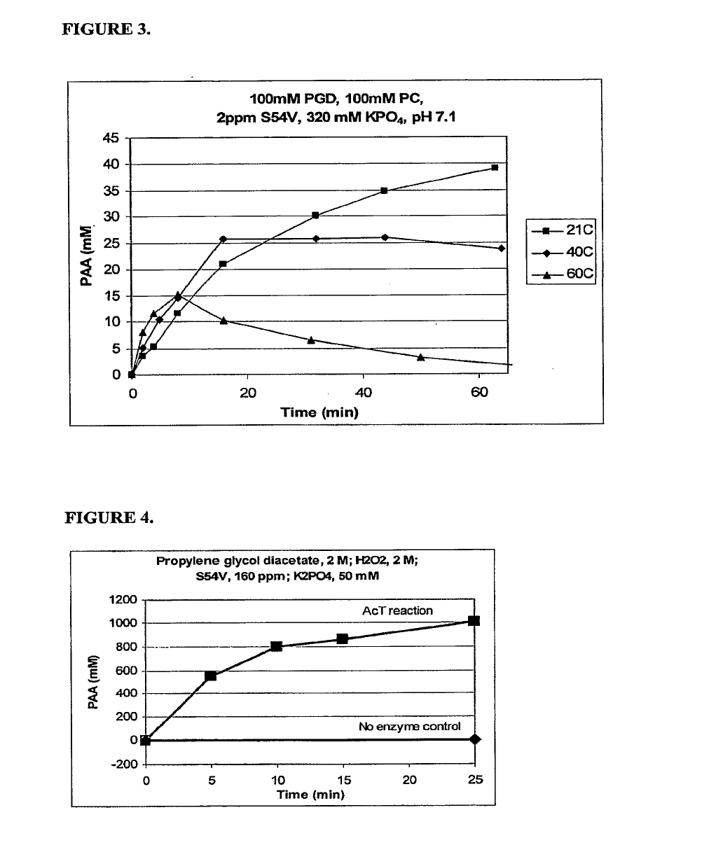

In these experiments, three reactions were prepared, consisting of 320 mM KPO₄ pH 7.1, 100 mM 1,2-propylene glycol diacetate (Aldrich), and 100 mM sodium percarbonate. The reactions were equilibrated at 21° C., 40° C. and 60° C., and then initiated by the addition of S54V variant to a final concentration of 2 ppm. The results are presented in FIG. 3. Samples were withdrawn at the times indicated in the Figure, and the concentration of PAA determined as described above. These results indicate that the enzyme system is functional at least up to 60° C.

### Example 7

### Generation of Concentrated Peracetic Acid by AcT

In this Example experiments conducted to determine the ability of AcT to generate a concentrated solution of peracetic acid. These experiments were conducted in order to address the potential benefit of preparing a concentrated peracetic acid solution which is suitable for dosing or dilution into different solutions for use. In this experiment the reaction contained 50 mM KPO₄, 2 M H₂O₂ (EM Science), 2 M 1,2-propylene glycol diacetate (Aldrich), and the S54V variant to a final concentration of 160 ppm. The reaction was vortexed occasionally to mix the reactants, as they were not miscible at this concentration. Mixing was conducted at room temperature. The results are shown in FIG. 4. Samples were diluted at the indicated times and the peracetic acid concentration was determined as described above.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

Having described the preferred embodiments of the present invention, it will appear to those ordinarily skilled in the art that various modifications may be made to the disclosed embodiments, and that such modifications are intended to be within the scope of the present invention.

Those of skill in the art readily appreciate that the present invention is well adapted to carry out the objects and obtain the ends and advantages mentioned, as well as those inherent therein. The compositions and methods described herein are representative of preferred embodiments, are exemplary, and are not intended as limitations on the scope of the invention. It is readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and sub generic groupings falling within the generic disclosure also form part of the invention. This includes the generic description of the invention with a proviso or negative limitation removing any subject matter from the genus, regardless of whether or not the excised material is specifically recited herein.

We claim:

1. An enzyme system for generation of peracid in aqueous solution, suitable for use in decontamination.

2. The enzyme system of claim 1, wherein said system comprises at least one ester substrate, at least one hydrogen peroxide source, and at least one acyl transferase.

3. The enzyme system of claim 1, wherein said peracid is selected from peracetic acid, pernonanoic acid, perproprionic, perbutanoic, perpentanoic, perhexanoic acid, peracids made from long chain fatty acids, and peracids made from short chain fatty acids.

4. The enzyme system of claim 2, further comprising at least one chemical hydrogen peroxide generation system, wherein said chemical hydrogen peroxide generation system comprises at least one chemical selected from sodium percarbonate, perborate, and urea hydrogen peroxide.

5. The enzyme system of claim 2, further comprising at least one enzymatic hydrogen peroxide generation system selected from oxidases and their corresponding substrates.

6. The enzyme system of claim 2, further comprising at least one enzymatic hydrogen peroxide generation system, wherein said enzymatic hydrogen peroxide generation system comprises at least one enzyme selected from glucose oxidase, sorbitol oxidase, hexose oxidase, choline oxidase, alcohol oxidase, glycerol oxidase, cholesterol oxidase, pyranose oxidase, carboxyalcohol oxidase, L-amino acid oxidase, glycine oxidase, pyruvate oxidase, glutamate oxidase, sarcosine oxidase, lysine oxidase, lactate oxidase, vanillyl oxidase, glycolate oxidase, galactose oxidase, uricase, oxalate oxidase, xanthine oxidase, and wherein said enzymatic hydrogen peroxide generating system further comprises at least one suitable substrate for said at least one enzyme.

7. The enzyme system of claim 1, further comprising at least one additional enzyme.

8. The enzyme system of claim 7, wherein said at least one additional enzyme is selected from proteases, cellulases, amylases, and microbial cell wall degrading enzymes.

9. The enzyme system of claim 1, wherein said at least one ester substrate is an alcohol ester.

10. The enzyme system of claim 1, further comprising at least one surfactant.

11. The enzyme system of claim 1, further comprising at least one detergent.

12. The enzyme system of claim 1, wherein said system is in a form selected from liquids, granules, foams, and emulsions.

13. A method for decontamination comprising the steps of:
a) providing an item in need of decontamination, and at least one system for generation of peracid in aqueous solution, suitable for use in decontamination;
b) exposing said item to said system under conditions such that the item is decontaminated.

14. The method of claim 13, wherein said exposing comprises exposing said item to said system under alkaline or acid pH conditions.

15. The method of claim 13, wherein said exposing comprises exposing said item to said system under neutral pH conditions.

16. The method of claim 13, wherein said exposing comprises exposing said item at high temperature.

17. The method of claim 13, wherein said system is in a form selected from liquids, granules, foams, and emulsions.

18. The method of claim 13, wherein said system comprises at least one ester substrate, at least one hydrogen peroxide source, and at least one acyl transferase.

19. The method of claim 13, wherein said peracid is selected from peracetic acid, pernonanoic acid, perproprionic, perbutanoic, perpentanoic, perhexanoic acid, peracids made from long chain fatty acids, and peracids made from short chain fatty acids.

20. The method of claim 18, further comprising at least one chemical hydrogen peroxide generation system selected from sodium percarbonate, perborate, and urea hydrogen peroxide.

21. The method of claim 18, wherein said system comprises at least one enzymatic hydrogen peroxide generation system selected from oxidases and their corresponding substrates.

22. The method of claim 18, wherein said system comprises at least one enzymatic hydrogen peroxide generation system is selected from glucose oxidase, sorbitol oxidase, hexose oxidase, choline oxidase, alcohol oxidase, glycerol oxidase, cholesterol oxidase, pyranose oxidase, carboxyalcohol oxidase, L-amino acid oxidase, glycine oxidase, pyruvate oxidase, glutamate oxidase, sarcosine oxidase, lysine oxidase, lactate oxidase, vanillyl oxidase, glycolate oxidase, galactose oxidase, uricase, oxalate oxidase, xanthine oxidase, and wherein said enzymatic hydrogen peroxide generating system further comprises at least one suitable substrate for said at least one enzyme.

23. The method of claim 13, further comprising at least one enzyme.

24. The method of claim 23, wherein said at least one enzyme is selected from proteases, amylases, cellulases, and microbial cell wall degrading enzymes.

25. The method of claim 13, wherein said at least one ester substrate is an alcohol ester.

26. The method of claim 13, further comprising at least one surfactant.

27. The method of claim 26, wherein said decontamination comprises decontaminating items contaminated by at least one toxin and/or at least one pathogen.

28. The method of claim 27, wherein said toxin is selected from botulinum toxin, anthracis toxin, ricin, scombroid toxin, ciguatoxin, tetrodotoxin, and mycotoxins.

29. The method of claim 27, wherein said pathogen is selected from bacteria, viruses, fungi, parasites, and prions.

30. The method of claim 29, wherein said at least one pathogen is selected from Bacillus spp., B. anthracis, Clostridium spp., C. botulinum, C. perfringens, Listeria spp., Staphylococcus spp., Streptococcus spp., Salmonella spp., Shigella ssp., E. coli, Yersinia spp., Y. pestis, Francisella spp., F tularensis, Camplyobacter ssp., Vibrio spp., Brucella spp., Cryptosporidium spp., Giardia spp., Cyclospora spp., and Trichinella spp.

31. The method of claim 13, wherein said item in need of decontamination is selected from hard surfaces, fabrics, food, feed, apparel, rugs, carpets, textiles, medical instruments, and veterinary instruments.

32. The method of claim 13, wherein said food is selected from fruits, vegetables, fish, seafood, and meat.

33. The method of claim 31, wherein said hard surfaces are selected from household surfaces and industrial surfaces.

34. The method of claim 33, wherein said household surfaces are selected from kitchen countertops, sinks, cupboards, cutting boards, tables, shelving, food preparation storage areas, bathroom fixtures, floors, ceilings, walls, and bedroom areas.

35. The method of claim 33, wherein said industrial surfaces are selected from food processing areas, feed processing areas, tables, shelving, floors, ceilings, walls, sinks, cutting boards, airplanes, automobiles, trains, and boats.

36. A method for decontamination comprising the steps of:
a) providing an item in need of decontamination, and at least one system for generation of peracid in aqueous solution, suitable for use in decontamination;
b) generating said peracid in aqueous solution; and
c) exposing said item to said peracid in aqueous solution under conditions such that the item is decontaminated.

37. The method of claim 36, wherein said exposing comprises exposing said item to said peracid alkaline or acid pH conditions.

38. The method of claim 36, wherein said exposing comprises exposing said item to said peracid under neutral pH conditions.

39. The method of claim 36, wherein said exposing comprises exposing said item to said peracid at high temperature.

40. The method of claim 36, wherein said system is in a form selected from liquids, granules, foams, and emulsions.

41. The method of claim 36, wherein said system comprises at least one ester substrate, at least one hydrogen peroxide source, and at least one acyl transferase.

42. The method of claim 36, wherein said peracid is selected from peracetic acid, pernonanoic acid, perproprionic, perbutanoic, perpentanoic, and perhexanoic acid and peracids made from long chain fatty acids, and peracids made from short chain fatty acids.

43. The method of claim 41, wherein said system comprises at least one chemical hydrogen peroxide system selected from sodium percarbonate, perborate, and urea hydrogen peroxide.

44. The method of claim 41, wherein said system comprises at least one enzymatic hydrogen peroxide generation system selected from oxidases and their corresponding substrates.

45. The method of claim 41, further comprising at least one enzymatic hydrogen peroxide generation system is selected from glucose oxidase, sorbitol oxidase, hexose oxidase, choline oxidase, alcohol oxidase, glycerol oxidase, cholesterol oxidase, pyranose oxidase, carboxyalcohol oxidase, L-amino acid oxidase, glycine oxidase, pyruvate oxidase, glutamate oxidase, sarcosine oxidase, lysine oxidase, lactate oxidase, vanillyl oxidase, glycolate oxidase, galactose oxidase, uricase, oxalate oxidase, xanthine oxidase, and wherein said enzymatic hydrogen peroxide generating system further comprises at least one suitable substrate for said at least one enzyme.

46. The method of claim 36, further comprising at least one enzyme.

47. The method of claim 46, wherein said at least one enzyme is selected from proteases, amylases, cellulases, and microbial cell wall degrading enzymes.

48. The method of claim 36, wherein said at least one ester substrate is an alcohol ester.

49. The method of claim 36, further comprising at least one surfactant.

50. The method of claim 36, wherein said decontamination comprises decontaminating items contaminated by at least one toxin and/or at least one pathogen.

51. The method of claim 50, wherein said toxin is selected from botulinum toxin, anthracis toxin, ricin, scombroid toxin, ciguatoxin, tetrodotoxin, and mycotoxins.

52. The method of claim 50, wherein said pathogen is selected from bacteria, viruses, fungi, parasites, and prions.

53. The method of claim 52, wherein said at least one pathogen is selected from Bacillus spp., B. anthracis, Clostridium spp., C. botulinum, C. perfringens, Listeria spp., Staphylococcus spp., Streptococcus spp., Salmonella spp., Shigella ssp., E. coli, Yersinia spp., Y pestis, Francisella spp., F. tularensis, Camplyobacter ssp., Vibrio spp., Brucella spp., Cryptosporidium spp., Giardia spp., Cyclospora spp., and Trichinella spp.

54. The method of claim 36, wherein said item in need of decontamination is selected from hard surfaces, fabrics, food, feed, apparel, rugs, carpets, textiles, medical instruments, and veterinary instruments.

55. The method of claim 36, wherein said food is selected from fruits, vegetables, fish, seafood, and meat.

56. The method of claim 55, wherein said hard surfaces are selected from household surfaces and industrial surfaces.

57. The method of claim 56, wherein said household surfaces are selected from kitchen countertops, sinks, cupboards, cutting boards, tables, shelving, food preparation storage areas, bathroom fixtures, floors, ceilings, walls, and bedroom areas.

58. The method of claim 56, wherein said industrial surfaces are selected from food processing areas, feed processing areas, tables, shelving, floors, ceilings, walls, sinks, cutting boards, airplanes, automobiles, trains, and boats.
